(19) United States
(12) Patent Application Publication
(10) Pub. No.: US 2017/0056575 A1
(43) Pub. Date: Mar. 2, 2017

(54) DIALYSIS CATHETER AND METHODS OF USE THEREOF

(71) Applicant: COOK MEDICAL TECHNOLOGIES LLC, Bloomington, IN (US)

(72) Inventors: Kalub Hahne, West Lafayette, IN (US); Emma Moran, West Lafayette, IN (US); Sarah Septiani, West Lafayette, IN (US); Amy Ham, West Lafayette, IN (US)

(73) Assignee: COOK MEDICAL TECHNOLOGIES LLC, Bloomington, IN (US)

(21) Appl. No.: 15/245,363

(22) Filed: Aug. 24, 2016

Related U.S. Application Data
(60) Provisional application No. 62/337,956, filed on May 18, 2016.
(60) Provisional application No. 62/210,511, filed on Aug. 27, 2015.

(51) Int. Cl. A61M 1/28; A61M 25/10; A61M 25/00

(52) U.S. Cl. CPC A61M 1/285; A61M 25/005; A61M 25/1002; A61M 25/1011; A61M 2205/0266; A61M 2025/1015

(57) ABSTRACT

The present invention relates generally to medical devices for use in peritoneal dialysis and to methods of using such devices. One aspect of the invention provides a device including an elongated tubular member having a lumen extending from a proximal end to a distal end and a first expandable member positioned around the elongated tubular member at a first position. A second expandable member is positioned around the elongated tubular member at a second position distal of the first position. The wall of the elongated tubular member between the first and second expandable members is free of an opening providing a fluid path between the lumen and an exterior of the device. The elongated tubular member may be reinforced along at least a portion of its length.

### CROSS-REFERENCE TO RELATED APPLICATIONS

This application claims the benefit of the filing dates of Provisional U.S. Patent Application Ser. No. 62/337,956, filed May 18, 2016 and 62/210,511, filed Aug. 27, 2015, which applications are hereby incorporated by reference.

### FIELD OF THE INVENTION

The present invention relates generally to medical devices for use in peritoneal dialysis and to methods of using such devices.

### BACKGROUND

End-stage renal disease (ESRD) occurs when the kidneys fail to support the body's needs in the last stage of chronic kidney disease (CKD). When this occurs, dialysis is required to help remove impurities and waste from the blood. The two types of dialysis that are most commonly performed are hemodialysis and peritoneal dialysis.

For hemodialysis patients, the creation of vascular access in the form of an arteriovenous fistula (AVF) or arteriovenous graft (AVG) is vital. However, these vascular access points need time to mature (6 weeks-4 months). During this period the patient still needs an outlet for dialysis to be performed, which is usually accomplished by utilizing the central venous catheter (CVC) as the initial dialysis access for hemodialysis. This type of catheter is problematic due to the high rate of infection, negatively impacting the patients' quality of life. CVC infections are dangerous, costly, and can cause complications that can lead to sepsis and death. Catheter related blood stream infections (CRESIs) caused by CVCs are also the leading source of nosocomial infections in the United States.

CVCs are also used for patients with acute kidney injury (AKI) or acute on early stage chronic kidney disease (ACKD). The disadvantage of CVC in this case is the decline of the estimated glomerular filtration rate (eGFR); it is even more rapid compared to hemodialysis using AVF.

In comparison with hemodialysis, peritoneal dialysis can better help preserve the residual renal function (RRF). The initiation of peritoneal dialysis is advantageous to let the kidney heal and restore its functions.

In current methods of peritoneal dialysis, a peritoneal dialysis catheter is inserted through the skin, subcutaneous fat, rectus muscle, and parietal peritoneum into the peritoneal cavity. A Dacron cuff is placed in the rectus muscle to encourage better attachment to the surrounding tissue. A healing period of 2-3 weeks is then needed in order to prevent inflammation and infection of the exit site and tunneled area. The movement of the peritoneal dialysis catheter can also cause leakage of dialysis solution and, consequently, infection of the exit site.

### SUMMARY

One aspect of the present invention provides a device including an elongated tubular member having a lumen extending from a proximal end to a distal end and a first expandable member positioned around the elongated tubular member at a first position. A second expandable member is positioned around the elongated tubular member at a second position distal of the first position. The wall of the elongated tubular member between the first and second expandable members is free of an opening providing a fluid path between the lumen and an exterior of the device.

In one embodiment, the elongated tubular member is reinforced along at least a portion of its length. The reinforcement may be a metallic reinforcement formed from, for example, stainless steel or a nickel-titanium shape-memory alloy, such as NITINOL. For example, the metallic reinforcement may be in the form of a metallic coil, a braid or a solid reinforcement.

In another embodiment, least one of the expandable members is a balloon. The balloon may be manufactured from, for example, a nylon, polyamide, polyolefin, polyester, polyurethane, fluoropolymer, polyethylene, polytetrafluoroethylene (PTFE), polyethyleneterepthalate (PET), polyvinyl chloride, latex, natural rubber, synthetic rubber, elastomer, silicone or mixtures or copolymers of at least two of these materials.

In yet another embodiment, the balloons are tapered from a broader end to a narrower end. The broader ends of the balloons may be positioned facing towards each other and towards the central portion of the device, i.e. the portion to be positioned traversing the abdominal wall. In yet another embodiment, the portions of the balloons positioned towards the central portion move towards each other as the balloons are inflated.

At least one of the expandable members may be slidably attached to the elongated tubular member to allow the distance between the balloons to be varied. The device may also include a locking mechanism configured to fix the slidably attached expandable member in position on the elongated tubular member.

In another embodiment, at least one of the expandable members is an expandable wire mesh structure. A portion of the structure may be covered, for example, by a covering including a silicone or polyethylene terephthalate.

In yet another embodiment, at least one of the expandable members is an expandable coil. The ends of the coil(s) may be tapered from a broader end to a narrower end. In some embodiments, the broader ends of the coils are positioned facing towards each other and the central portion. A portion of the coil(s) may be covered, for example, by a covering including a silicone or polyethylene terephthalate.

In another embodiment, at least one of the expandable members is a multi-winged malecot including at least two wings. A portion of the malecot(s) may be covered, for example, by a covering including a silicone or polyethylene terephthalate.

In yet another embodiment at least one of the expandable members includes a plurality of arms each having a first end and a second end, where the first end attaches to the elongated tubular member and where the second end extends radially away from the axis of the shaft when in the expanded state.

Another aspect of the invention provides a method for performing peritoneal dialysis. In one embodiment, the method includes inserting the distal end of a device as disclosed herein through an opening in the wall of the abdomen and expanding the expandable member(s) to an extent sufficient to position portions of the expandable member(s) against the wall of the abdomen and seal and fluid path in the opening exterior to the elongated tubular member. A dialysate fluid is then introduced into the abdomen through the lumen and maintained in the abdomen for a time sufficient for waste products diffuse into the fluid from blood vessels surrounding the abdomen. The fluid is then removed from the abdomen through the lumen.

### BRIEF DESCRIPTION OF THE DRAWINGS

The invention may be more fully understood by reading the following description in conjunction with the drawings, in which:

FIG. 1 is a schematic illustration of one embodiment of a catheter of the present invention. The catheter includes two expandable balloons, which are illustrated in an expanded configuration.

FIG. 2 is a schematic illustration of the embodiment of FIG. 1 in position bridging the abdominal wall.

FIG. 3 is a schematic illustration of another embodiment of a catheter of the present invention. The catheter includes two expandable tapered balloons. The balloons are illustrated in an expanded configuration.

FIG. 4 is a schematic illustration of yet another embodiment of a catheter of the present invention. The catheter includes two expandable wire mesh structures, which are illustrated in an expanded configuration.

FIG. 5 is a schematic illustration of another embodiment of a catheter of the present invention. The catheter includes two expandable coils, which are illustrated in an expanded configuration.

FIG. 6 is a schematic illustration of another embodiment of a catheter of the present invention. The catheter includes two expandable multi-winged malecots, which are illustrated in an expanded configuration.

FIG. 7 is a schematic illustration of an embodiment of an expandable structure suitable for use in a catheter of the present invention. The expandable structure includes a number of arms attached at one end to the catheter body. The petal-shaped arms are illustrated in their expanded configuration.

FIG. 8 is a schematic illustration of another embodiment of an expandable structure suitable for use in a catheter of the present invention. The expandable structure includes a number of arms attached at one end to the catheter body. The arms are illustrated in their expanded configuration and form a star-shaped structure.

### DEFINITIONS

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. The materials, methods, and examples disclosed herein are illustrative only and not intended to be limiting.

As used herein the terms “comprise(s),” “include(s),” “having,” “has,” “can,” “contain(s),” and variants thereof, are intended to be open-ended transitional phrases, terms, or words that do not preclude the possibility of additional acts or structures. The present invention also contemplates other embodiments “comprising,” “consisting of” and “consisting essentially of,” the embodiments or elements presented herein, whether explicitly set forth or not.

As used herein the terms “proximal” and “distal” are used to describe the opposing axial ends of the device, as well as the axial ends of various component features of the device. The term “proximal” is used in its conventional sense to refer to the end of the device (or component of the device) that is closest to the medical professional during use of the device. The term “distal” is used in its conventional sense to refer to the end of the device (or component of the device) that is initially inserted into the patient, or that is farer from the medical professional during use of the device.

### DETAILED DESCRIPTION

For the purposes of promoting an understanding of the principles of the invention, reference will now be made to the embodiments illustrated in the drawings and specific language will be used to describe the same. It will nevertheless be understood that no limitation of the scope of the invention is thereby intended, and alterations and modifications in the illustrated device, and further applications of the principles of the invention as illustrated therein are herein contemplated as would normally occur to one skilled in the art to which the invention relates.

### Devices for Performing Peritoneal Dialysis

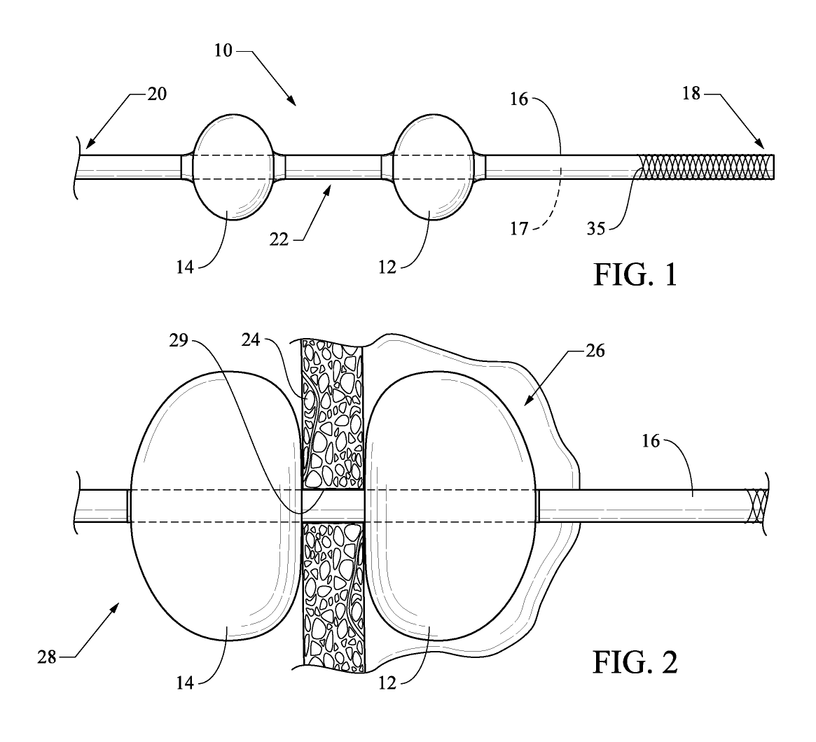

One aspect of the present invention provides a medical device for performing peritoneal dialysis. Referring now to FIG. 1, there is illustrated a portion of one embodiment of such a device. Catheter 10 includes elongated tubular member 16 having lumen 17 extending from proximal end 20 to distal end 18. First expandable member 14 is positioned around a surface of elongated tubular member 16 at a first position. Second expandable member 12 is positioned around elongated tubular member 16 at a second position, which is distal of the first position. The wall of elongated tubular member 16 at central portion 22 between the two expandable members is free of an opening providing a fluid path between lumen 17 and an exterior of the device. In certain embodiments, at portion of elongated tubular member 16, for example distal portion 35, may be reinforced as is disclosed herein.

In the embodiment illustrated in FIG. 1, the expandable members may be inflatable balloons. In such an embodiment, the device also includes at least one inflation tube (not illustrated) to provide for inflation of the balloons. For example, the device may include separate inflation tubes for each of the balloons. Alternatively, a single inflation tube may provide for the inflation of both balloons. The inflation tubes will typically extend from the proximal region of the device to the inflatable balloons and include an inflation lumen in fluid contact with the interior of the balloon(s). In one embodiment, the inflation tubes are contained within lumen 17 of elongated tubular member 16. For example, elongated tubular member 16 may have two or three lumens; a larger lumen for dialysis fluid to pass through and one or two smaller lumens that provide for inflation of the balloons.

Turning now to FIG. 2. This figure illustrates the typical placement of the device of FIG. 1 during peritoneal dialysis. Initially, the distal end of the device is inserted through opening 29 in the abdominal cavity wall 24 such that distal expandable member 12 is positioned within the abdominal cavity 26 and proximal expandable member 14 is at region 28, i.e. exterior to the patient or at least outside the wall of the abdominal cavity. Both of the expandable members, or at least the distal expandable member, will normally be in a deflated or compressed state during this stage of the procedure. The expandable member(s) are then inflated. For example, the expandable members may be inflated through the inflation tube(s) with gas or fluid.

One expandable member will typically be situated on the posterior side of the rectus muscle inside the peritoneal cavity. The second expandable member will typically be anterior to the rectus muscle and underneath the subcutaneous fat and skin. Inflation of the expandable members sandwiches the rectus muscle between the expandable members and provides sealing and mechanical immobilization so that the catheter can be used immediately for peritoneal dialysis.

In one embodiment, the space between the two expandable members may contain a material to encourage cellular ingrowth and affixing into the rectus sheath. In other embodiments, the region 22 between the two expandable members may include a cuff to assist in sealing of the entry point.

After sealing opening 29, the catheter allows the immediate initiation of peritoneal dialysis by preventing the leakage of dialysate fluid and associated infections. The dialysate fluid is delivered from the proximal end of elongated member 16 to the distal end of the device and then into the abdominal cavity. Hence, lumen 17 provides a leak fee pathway from the exterior into the abdomen of the patient.

In some embodiments, after the rectus muscle has infiltrated and adhered to the biomaterial the balloons can be deflated. If the catheter does not have a material for tissue ingrowth the balloons can be deflated for the ease of removal of the catheter if the catheter is used for temporary dialysis treatment.

The balloons of the device may be manufactured from any materials typically used for the manufacture of inflatable balloons. For example, the balloons may include materials such as a nylon, polyamide, polyolefin, polyester, polyurethane, fluoropolymer, polyethylene, polytetrafluoroethylene (PTFE), polyethyleneterepthalate (PET), polyvinyl chloride, latex, natural rubber, synthetic rubber, elastomer, silicone or mixtures and copolymers or two of more of there materials.

In certain embodiments, one of the expandable members may be slidably attached to elongated tubular member 16. This configuration allows for the separation of the expandable members to be varied and can provide for improved sealing when the members are expanded. In these embodiments, the movable member may be provided with a locking mechanism to allow the balloon to be locked in place on elongated tubular member 16 at the required position.

In other embodiments, the expandable members may be balloons that are shaped such that the portions of the balloons adjacent to the abdomen wall move towards each other into the central portion of the device between the balloons as they are inflated. In such an embodiment, the abdominal wall is tightly sandwiched between the two balloons, providing for improved sealing of the catheter entry opening

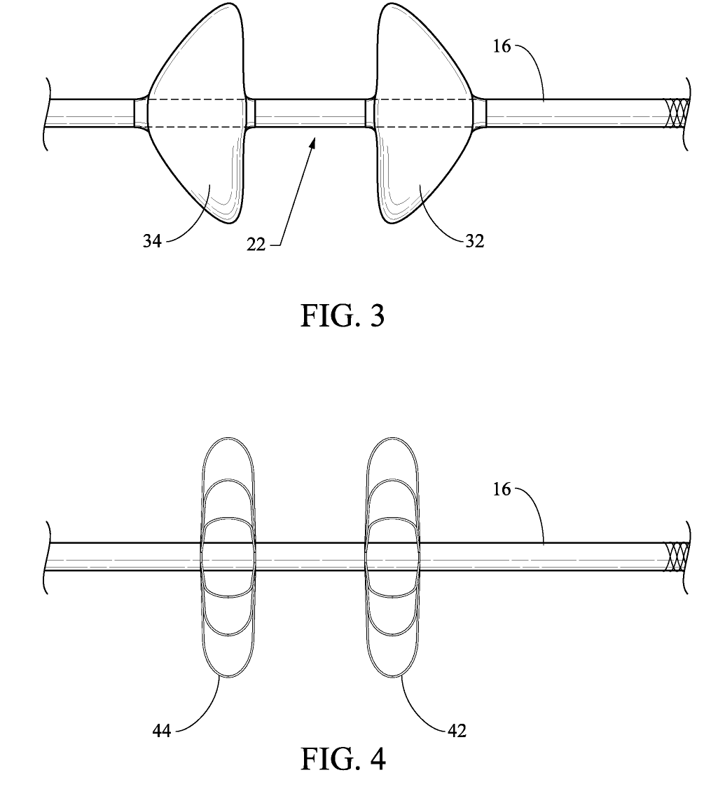

FIG. 3 illustrates another embodiment of the invention. In this embodiment, the expandable members are also balloons. However, in this embodiment the balloons are tapered from a broader end to a narrower end. The broader ends of the balloons are positioned facing towards each other and towards the central portion of the device between the balloons. In this embodiment, one or both of the balloons may be slidably attached to the elongated tubular member so that the separation between the balloons may be varied and the balloon(s) locked in position to provide for a required separation of the balloons. In other embodiments, the portions of the balloons adjacent to the abdomen wall may move towards each other into the central portion of the device between the balloons as they are inflated.

Turning how to FIG. 4. In this embodiment, the expandable members 44 and 42 are formed from an expandable mesh. The mesh may be formed from a metal, for example, stainless steel or a super-elastic alloy and/or a shape-memory alloy, e.g., a nickel-titanium shape-memory alloy such as NITINOL. In certain embodiments, the mesh may be covered to provide for improved sealing of the abdominal entry point. For example, the mesh may be covered with a material such as a silicone or polyethylene terephthalate.

Typically, the mesh will be biased to the expanded configuration. In such embodiments, the device may be surrounded by a delivery sheath during entry of the device into the abdominal cavity. The sheath may maintain the expandable mesh in a compressed configuration during placement of the device. Following delivery, the sheath may be removed allowing for expansion of the mesh structure(s).

As with the embodiments including inflatable balloons, one or both of the mesh structures may be slidable attached to the elongated tubular member so that the separation between the structures may be varied and the structures locked in position to provide a required separation of the structures. In other embodiments, the portions of the mesh structures facing to the abdomen wall may move towards each other into the central portion of the device between the mesh structures expand. For example, the end(s) of the mesh structure(s) farthest from the abdominal wall may be attached to the elongated tubular member while the end(s) closest to the abdomen wall may be free to slide towards the wall upon expansion.

In certain embodiments, the mesh structure(s) are made by bending or braiding metal wire. The wire ends may be gathered around the circumference of the elongated tubular member. The end farthest from the abdomen wall is affixed to the elongated tubular member whereas the end nearest to the abdomen is attached to a structure, such as a ring, which is able to move with respect to the fixed end.

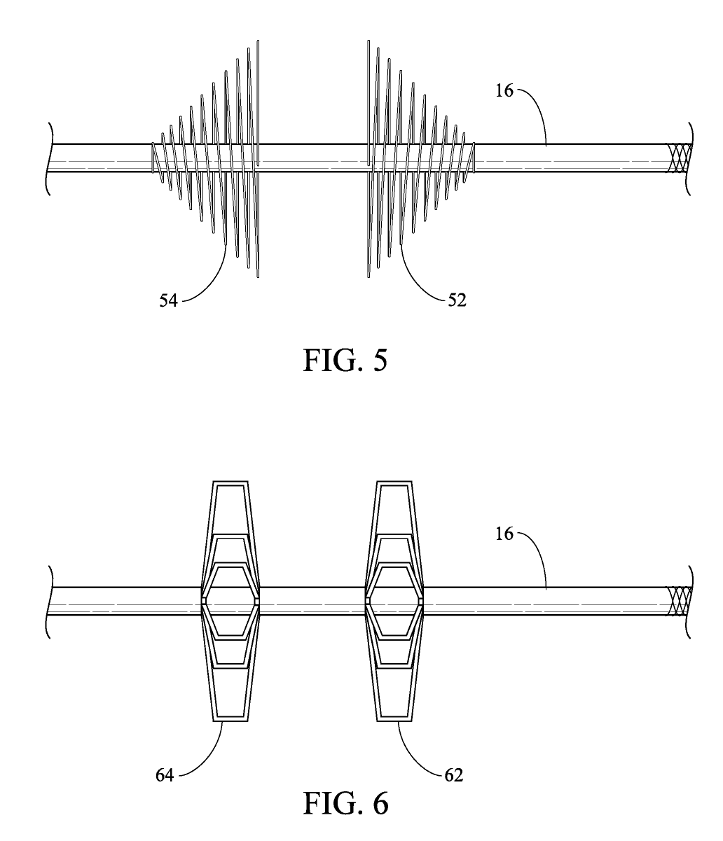

FIG. 5 illustrates yet another embodiment of the invention. Here, expandable coils 52 and 54 replace the wire mesh structures. Portions of the expandable coils may be provided with a covering to provide for improved sealing of the abdominal entry opening. In one embodiment, ends of the coils are tapered from a broader end to a narrower end. Typically, the broader ends of the coils are positioned facing towards each other and the central portion of the device, as is illustrated in FIG. 5. One or both of the expandable coils may be slidably attached to the elongated tubular member so that the separation between the structures may be varied and the coils locked in position to provide a required separation of the coils.

FIG. 6 illustrates yet another embodiment of the invention. This this embodiment the expandable members are multi-winged malecots 62 and 64 including at least two wings. Again, the expandable members may be at least partially covered to provide for improved sealing and at least one of the malecots may be slidably attached to the elongated tubular member as described above.

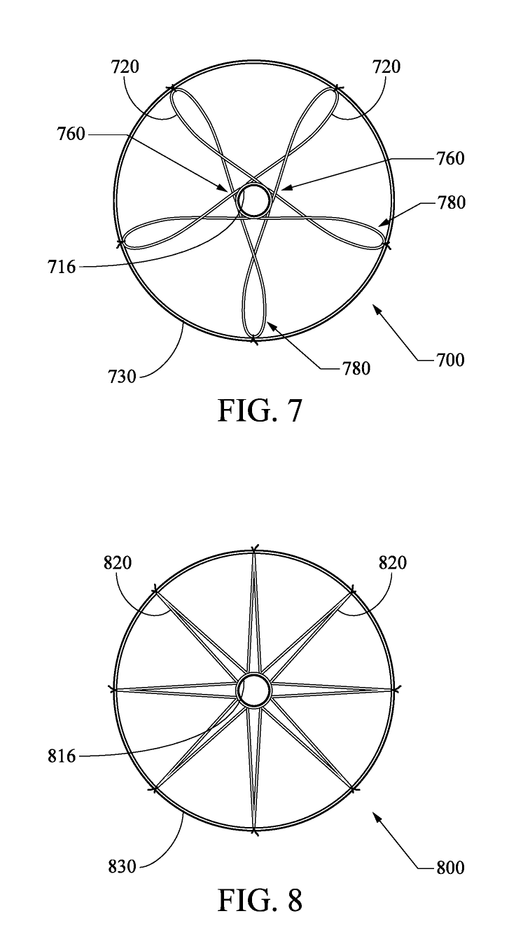

Turning now to FIG. 7, here there is illustrated another embodiment of an expandable structure suitable for use with a catheter of the present invention. The structure is illustrated in an expanded state in a view taken along the longitudinal axis of catheter 716. Expandable structure 700 includes five petal-shaped arms 720. One end 760 of each arm 720 attaches to the catheter shaft while the other end 780 extends radially away from the axis of the shaft when in the expanded state.

In a compressed state, the end 780 of each arm 720 lies adjacent to catheter 716. Typically, each arm 720 is constrained in the compressed state by insertion within the lumen of a delivery sheath. For example, the arms 720 for an expandable structure positioned to reside inside the abdominal cavity may extend proximally from attachment ends 760 when constrained by a delivery sheath. When the sheath is removed, the ends 780 move away from catheter and take up the expanded configuration. The expandable structure may then be moved into positioned against the inside of the abdominal wall so as to seal the entry point of the catheter from the inside.

If a similar expandable structure is positioned to reside outside of the abdominal wall, the arms of this expandable structure extend distally (towards the outside of the abdominal wall) when in the compressed configuration. When released from the constraint of a delivery sheath, the arms move away from the axis of the catheter and take up the expanded configuration.

Although five arms are illustrated in FIG. 7, structures including 3, 4, 6, 7, 8, 9 or more arms are also within the scope of the present invention. Each arm may be formed from a thin wire bent into the shape illustrated in FIG. 7. Alternatively, the arms may be solid and may be, for example, cut from sheet material. FIG. 8 shows a similar view of an expandable structure including 8 arms 820, each attaching to catheter shaft 816. Here, eight pointed arms 820 are attached to the shaft of catheter 816.

The arms 720 or 820 will typically be biased to conform to the expanded configuration when not constrained against the wall of the catheter. This may be achieved by forming the arms from a metal, such as stainless steel or a shape-memory alloy, e.g., a nickel-titanium shape-memory alloy such as NITINOL.

In some embodiments, the arms may be covered by graft material 730 or 830. The graft may be formed form a polymer material, such as a silicone or polyethylene terephthalate, and may be attached to the arms by, for example, sutures.

In certain embodiments, the device may include at least one cuff positioned on the wall of the elongated tubular member at the central portion. For example, the device may include a cuff over a portion of the central portion between the two expandable members. The cuff may provide for improved sealing of the catheter entry opening. The cuff may be formed from a material including, for example, polyethylene terephthalate (e.g. DACRON) or another biocompatible polymer material.

In other embodiments, the cuff includes a remodelable material. Such a remodelable material is capable of remodeling or promoting cell growth and/or promoting regrowth and healing of damaged tissue structures. The presence of such a remodelable material may encourage cellular ingrowth and affixing of the catheter into the rectus sheath. This may help stabilize the catheter and provide improved sealing of the opening. In other embodiments, the remodelable material is present on the surface of the elongated member or is incorporated into the structure of the elongated member.

The remodelable material can be, for example, extra cellular material (ECM), small intestine submucosa (SIS), remodelable or collagenous foam, foamed ECM, lyophilized SIS or vacuum pressed SIS. One non-limiting example of a suitable remodelable material is the SURGISIS™. BIODESIGN™, commercially available from Cook Incorporated, Bloomington, Ind. Another suitable remodelable material is the graft prosthesis material described in U.S. Pat. No. 6,206,931 to Cook et al., incorporated herein by reference.

Another embodiment of the invention provides a device having a single expandable member, such as those expandable members disclosed herein. For example, the single expandable member may be a balloon, a tapered balloon, an expandable mesh structure, an expandable coil, a multi-winged malecot including at least two wings or in the form of a plurality of arms as illustrated in FIGS. 7 and 8. Typically, in these embodiments, the second expandable member is replaced by a cuff sized and shaped to press against the opposite side of the abdominal wall when the expandable member is expanded and to sandwich the abdominal wall between the expandable member and the cuff. Here, the single expandable member many be positioned interior or exterior to the abdominal wall.

In embodiments including only one expandable member, the expandable member may be positioned inside the abdominal cavity during the dialysis procedure. The cuff is positioned outside the abdomen and against the abdomen wall. As the expandable member is expanded, a portion of the expandable member presses against the inside of the abdomen wall resulting in the cuff being pulled towards the outside surface of the wall and resulting in the sealing of the catheter entry opening by the expandable member.

However, the present invention also includes those embodiments having a single expandable member placed proximally of the cuff. In these embodiments, the expandable member remains outside the abdominal cavity and the cuff is place inside the abdominal cavity during the dialysis procedure. The invention also encompasses embodiments including two but differing expandable members. For example, the device may include any two of a balloon, a tapered balloon, an expandable mesh, an expandable coil and a multi-winged malecot.

In certain embodiments, the elongated tubular member of the catheter is reinforced along at least a portion of its length. In some embodiments, the reinforcement may provide for stabilization of the catheter within the abdominal cavity. The density (degree) of reinforcement may be constant or may vary along the length of the elongated tubular member. In some embodiments only portions of the elongated tubular body include a reinforcing member. In other embodiments, the degree of reinforcement may be varied by, for example, varying the thickness of the reinforcing member. If the reinforcing member is a coil or a similar structure, the degree of reinforcement may also be varied by varying the pitch of the coil along the length of the elongated tubular member.

The portion of the elongated tubular member near the proximal tip of the catheter may include a reinforced region to discourage migration while the portion proximal of this region and distal of the expandable structures may have lesser or no reinforcement such that the flexibility of this portion of the elongated tubular member is maintained. The distal region of the catheter may be curved in the shape of a “pig-tail” to provide for additional stabilization of the catheter.

In another embodiment, the portions of the elongated tubular member around (distal and/or proximal of) the expandable structures may be reinforced with lesser of no reinforcement in the region distal of the region around the distal expandable structure. In yet other embodiments, portions of the catheter shaft around (distal or proximal of) the expandable structures and in the region around the distal end of the expandable structure are reinforced while the region between these reinforcements has lesser or no reinforcement.

The reinforcement may be a metallic reinforcement, for example, formed from a material such as stainless steel or a nickel-titanium shape-memory alloy, such as NITINOL. The reinforcement may be in the form of a metallic coil, a braid or a solid reinforcement. Combinations of such reinforcements may be present at the same or at different portions of the catheter shaft. In some embodiments, the reinforcement is at least partially embedded within the wall of the elongated tubular member. For example, the wall may be formed from a silicone and the reinforcement at least partially embedded within the silicone wall.

Reinforcement of the wall may allow for a reduction in the thickness of the wall of the elongated tubular member. In various embodiments, the thickness of the wall along at least a portion of the elongated tubular member is between 2 mm and 0.25 mm, or 2 mm and 0.5 mm, or 1.5 mm and 0.5 mm or 1.0 mm and 0.5 mm or 2 mm and 1.0 mm or 0.25 mm and 0.5 mm, or 0.25 mm and 0.4 mm, or 0.25 mm and 0.3 mm.

Another aspect of the present invention provides a method for performing peritoneal dialysis. In one embodiment of the method, the distal end of a catheter device as disclosed above is inserted through an opening in a wall of the abdominal wall of a patient requiring dialysis. If the device includes two expandable members, the distal member is placed within the abdominal cavity and the proximal member on the outside of the abdominal wall. In those embodiments where the device includes only one expandable member, the expandable member is placed on one side of the abdominal wall and the cuff is positioned to sandwich the abdominal wall between the cuff and the expandable member. During this stage of the procedure, the expandable member(s), or at least the distal expandable member, will typically be in a deflated or compressed configuration. For example, the expandable member(s) may be confined within the lumen of a delivery sheath.

The expandable member(s) are the expanded by, for example, inflation, if they are balloons or by removing a covering sheath in the case of an expandable mesh, coil or malecot. Inflation of the expandable member(s) will sandwich the abdominal wall between the members or the single member and a cuff and seal the catheter entry opening and stabilize the catheter.

A volume of fluid is introduced into the abdomen through the lumen of the elongated member. The expandable member(s) provide sealing of the catheter entry opening and allow for peritoneal dialysis to commence immediately. Typically, the volume of fluid introduced is about 2 to 3 liters and the fluid is introduced over a period of about ten to fifteen minutes. The fluid remains in the abdominal cavity and waste products diffuse across the peritoneum from the underlying blood vessels and into the fluid. After a variable period of time depending on the treatment, typically 4-6 hours, the fluid is removed through the lumen of the device and replaced. This process can occur automatically while the patient is sleeping (automated peritoneal dialysis), or during the day by keeping two to three liters of fluid in the abdomen at all times, exchanging the fluids four to six times per day (continuous ambulatory peritoneal dialysis).

The catheter may be used as a temporary measure to provide dialysis to maintain blood homeostasis, while waiting for another form of dialysis treatment to begin, to allow the kidneys to recover from trauma, or while waiting for hemodialysis access points to heal. In other embodiments, the catheter may be used permanently for peritoneal dialysis.

The fluid used typically contains sodium, chloride, lactate or bicarbonate and a high percentage of glucose to ensure hyperosmolarity. The amount of dialysis that occurs depends on the volume of the fluid, the regularity of the exchange and the concentration of the fluid. The presence of the catheter presents a risk of peritonitis due to the potential to introduce bacteria to the abdomen. Peritonitis may be treated through the direct infusion of antibiotics into the peritoneum in the fluid.

While preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

We claim:

1. A device comprising:
an elongated tubular member having a lumen extending from a proximal end to a distal end and comprising a wall;
a first expandable member positioned around a surface of the elongated tubular member at a first position;
a second expandable member positioned around the elongated tubular member at a second position, wherein the second position is distal of the first position;
wherein the wall of the elongated tubular member at a central portion between the first expandable member and the second expandable member is free of an opening providing a fluid path between the lumen and an exterior of the device.

2. The device of claim 1, wherein the wall comprises a metallic reinforcement along at least a portion of its length.

3. The device of claim 2, wherein the metallic reinforcement comprises at least one of a metallic coil, a braided reinforcement and a solid reinforcement.

4. The device of claim 2, wherein the wall of the device comprises a silicone and wherein the metallic coil is at least partially embedded in the silicone.

5. The device of claim 2, wherein the metallic reinforcement is greater near the distal end and decreases towards the second expandable member.

6. The device of claim 2, wherein the metallic reinforcement comprises a material selected from the group consisting of stainless steel and a nickel-titanium shape-memory alloy.

7. The device of claim 1, wherein at least one of the first expandable member and the second expandable member is a balloon and wherein the device further comprises an inflation tube extending from a proximal region of the device to the balloon.

8. The device of claim 7, wherein the balloon comprises a material selected from the group consisting of a nylon, polyamide, polyolefin, polyester, polyurethane, fluoropolymer, polyethylene, polytetrafluoroethylene (PTFE), polyethyleneterepthalate (PET), polyvinyl chloride, latex, natural rubber, synthetic rubber, elastomer, silicone and mixtures and copolymers thereof

9. The device of claim 7, wherein the first expandable member and the second expandable member are balloons, wherein the balloons are tapered from a broader end to a narrower end, and wherein the broader ends of the balloons are positioned facing towards each other and the central portion.

10. The device of claim 1, where at least one of the first expandable member and the second expandable member is slidably attached to the elongated tubular member.

11. The device of claim 1, wherein at least one of the first expandable member and the second expandable member is an expandable wire mesh structure.

12. The device of claim 11, further comprising a covering over at least a portion of the at least one expandable wire mesh structure.

13. The device of claim 1, wherein at least one of the first expandable member and the second expandable member is an expandable coil.

14. The device of claim 13, further comprising a covering over at least a portion of the expandable coil.

15. The device of claim 1, wherein at least one of the first expandable member and the second expandable member is a multi-winged malecot including at least two wings.

16. The device of claim 15, further comprising a covering over at least a portion of the multi-winged malecot.

17. The device of claim 1, further comprising a cuff positioned on the wall of the elongated tubular member at the central portion.

18. The device of claim 1, wherein at least one of the first expandable member and the second expandable member comprises a plurality of arms each having a first end and a second end, wherein the first end attaches to the elongated tubular member and wherein the second end extends radially away from the axis of the shaft when in the expanded state.

19. A device comprising:
an elongated tubular member having a lumen extending from a proximal end to a distal end;
a first member positioned around a surface of the elongated tubular member at a first position;
a second expandable member positioned around the elongated tubular member at a second position, wherein the second position is distal of the first position, wherein the second expandable member comprises a plurality of arms each having a first end and a second end, wherein the first end attaches to the elongated tubular member and wherein the second end extends radially away from the axis of the shaft when in the expanded state;
wherein the wall of the elongated tubular member at a central portion between the first member and the second expandable member is free of an opening providing a fluid path between the lumen and an exterior of the device.

20. A method for performing peritoneal dialysis, comprising: inserting a distal end of a device through an opening in a wall of the abdomen, wherein the device comprises:
an elongated tubular member having a lumen extending from a proximal end to the distal end;
a first expandable member positioned around a surface of the elongated tubular member at a first position;
a second expandable member positioned around the elongated tubular member at a second position, wherein the second position is distal of the first position; and wherein the device is inserted so as the second expandable member is positioned within the abdomen and the first expandable member is positioned outside the abdomen;
expanding the first and second expandable members to an extent sufficient to position portions of the first and second expandable members against the wall of the abdomen and seal and fluid path in the opening exterior to the elongated tubular member;
introducing a fluid into the abdomen through the lumen;
maintaining the fluid in the abdomen for a time sufficient for waste products diffuse into the fluid from blood vessels surrounding the abdomen; and
removing the fluid from the abdomen through the lumen.
